(19) United States
(12) Patent Application Publication
(10) Pub. No.: US 2010/0258138 A1
(43) Pub. Date: Oct. 14, 2010

(54) RADIOLABELED 1-ACETATE PET IMAGING FOR RADIOTHERAPY IN HEAD AND NECK CANCER

(76) Inventors: Jens Sorensen, Uppsala (SE); Bengt Langstrom, Uppsala (SE); Aijun Sun, Umea (SE); Silvia Johansson, Umea (SE)

Correspondence Address: GE HEALTHCARE, INC., IP DEPARTMENT 101 CARNEGIE CENTER, PRINCETON, NJ, 08540-6231, US

(21) Appl. No.: 12/376,463

(22) PCT Filed: Aug. 22, 2007
(86) PCT No.: PCT/IB07/02412

§ 371 Date: Jul. 1, 2010

Related U.S. Application Data
(60) Provisional application No. 60/823,111, filed on Aug. 22, 2006.

(51) Int. Cl. A61B 6/00; A61K 51/04; C07C 53/10

(52) U.S. Cl. 128/898; 424/1.81; 424/1.89; 562/607

(57) ABSTRACT

The present invention provides methods of using optimal PET tracers for diagnosing head and neck cancer. Methods for in vivo imaging uses of the PET tracers that are suitable for uses in radiation therapy (RT) in head and neck cancer and evaluation of salivary gland function are also provided. A pharmaceutical comprising the PET tracer and a kit for the preparation of the pharmaceutical are provided as well.

### FIELD OF THE INVENTION

The present invention relates to the development of Positron Emission Tomography (PET) tracers that could be used for imaging for radiotherapy in head and neck cancer. The present invention further relates to methods for in vivo imaging uses of the PET tracers that are suitable for uses in radiation therapy (RT) in head and neck cancer and evaluation of salivary gland function. A pharmaceutical comprising the compound and a kit for the preparation of the pharmaceutical are also provided.

### BACKGROUND OF THE INVENTION

Tracers labeled with short-lived positron emitting radionuclides (e.g. ¹⁸F and ¹¹C) are the positron-emitting nuclide of choice for many receptor imaging studies. Accordingly, radiolabeled ligands have great clinical potential because of their utility in Positron Emission Tomography (PET) to quantitatively detect and characterize a wide variety of diseases.

Head and neck squamous cell carcinoma is curable when diagnosed at early stage (Panje W R, Namon A J, Vokes E, Haraf D J, Weichselbaum R R. Surgical management of the head and neck cancer patient following concomitant multimodality therapy. Laryngoscope 1995; 105:97-101). Both accurate diagnosis and staging of the tumors are important for prognosis and determination of treatment strategies. Conventional anatomic imaging techniques, such as computed tomography (CT), magnetic resonance imaging (MRI) and ultrasonography, are routinely used for evaluation of size and local tumor extend. However, there are inherent limitations associated with all these techniques (Vermeersch H, Loose D, Ham H, Otte A, Van de Wiele C. Nuclear medicine imaging for the assessment of primary and recurrent head and neck carcinoma using routinely available tracers. Eur J Nucl Med Mol Imaging 2003; 30:1689-700).

Positron emission tomography (PET) may improve the ability to noninvasively detect the biological characteristics of the tumors. ¹⁸F-fluoro-2-deoxy-D-glucose (FDG) PET has been widely applied for staging of the tumor, distinguishing tumor recurrence and predicting treatment response in head and neck cancer (Greven K M. Positron-emission tomography for head and neck cancer. Semin Radiat Oncol 2004; 14:121-9, Schwartz D L, Ford E C, Rajendran J, Yueh B, Coltrera M D, Virgin J, et al. FDG-PET/CT-guided intensity modulated head and neck radiotherapy: a pilot investigation. Head Neck 2005; 27:478-87, Avril N E, Weber W A. Monitoring response to treatment in patients utilizing PET. Radiol Clin North Am 2005; 43:189-204). PET is also increasing its use in delineation of gross tumor volume (Paulino A C, Johnstone P A. FDG-PET in radiotherapy treatment planning: Pandora's box? Int J Radiat Oncol Biol Phys 2004; 59:4-5).

FDG is an analog of glucose with high uptake in malignant cells, due to increased energy requirement (Strauss L G, Conti P S. The applications of PET in clinical oncology. J Nucl Med 1991; 32:623-48). However, FDG is not a specific tumor marker. It accumulates in inflammatory tissues and it also has limitations in finding well differentiated tumors (Goerres G W, Von Schulthess G K, Hany T F. Positron emission tomography and PET CT of the head and neck: FDG uptake in normal anatomy, in benign lesions, and in changes resulting from treatment. A J R Am J Roentgenol 2002; 179:1337-43, Delbeke D, Coleman R E, Guiberteau M J, Brown M L, Royal H D, Siegel B A, et al. Procedure guideline for tumor imaging with 18F-FDG PET/CT 1.0. J Nucl Med 2006; 47:885-95). Development of new tracers for improving the efficiency of PET imaging in head and neck cancer is therefore warranted.

Several recent studies have demonstrated that ¹¹C-acetate (ACE) might be a useful tracer for a few cancer types, such as lung cancer, hepatocellular carcinoma, renal cancer, prostate cancer and astrocytomas (Higashi K, Ueda Y, Matsunari I, Kodama Y, Ikeda R, Miura K, et al. 11C-acetate PET imaging of lung cancer: comparison with 18F-FDG PET and 99 mTc-MIBI SPET. Eur J Nucl Med Mol Imaging 2004; 31:13-21, Ho C L, Yu S C, Yeung D W. 11C-acetate PET imaging in hepatocellular carcinoma and other liver masses. J Nucl Med 2003; 44:213-21, Fricke E, Machtens S, Hofmann M, van den Hoff J, Bergh S, Brunkhorst T, et al. Positron emission tomography with 11 C-acetate and 18F-FDG in prostate cancer patients. Eur J Nucl Med Mol Imaging 2003; 30:607-11, Shreve P, Chiao P C, Humes H D, Schwaiger M, Gross M D. Carbon-11-acetate PET imaging in renal disease. J Nucl Med 1995; 36:1595-601, Liu R S, Chang C P, Chu L S, Chu Y K, Hsieh H J, Chang C W, et al. PET imaging of brain astrocytoma with 1-(11)C-acetate. Eur J Nucl Med Mol Imaging 2006; 33:420-7). Ho et al (Ho C L, Yu S C, Young D W. 11C-acetate PET imaging in hepatocellular carcinoma and other liver masses. J Nucl Med 2003; 44:213-21) reported that well-differentiated hepatocellular carcinoma displayed increased ACE uptake and minimal FDG uptake. These findings indicated that ACE and ¹⁸F-acetate may have a high sensitivity and specificity as a radiotracer complementary to FDG in the PET imaging of hepatocellular carcinoma.

The present knowledge of ACE-PET and ¹⁸F-acetate-PET in head and neck cancer is, however, sparse. Head and neck cancer is a lethal malignancy for which combinations of surgery, chemotherapy and/or radiation therapy (RT) are used for curative intent. There is a growing need for developing new molecular imaging technologies with high sensitivity and specificity in this field. First, optimal staging of this cancer is not reached in all patients using CT, MRI or FDG-PET. Secondly, using standard RT approaches, the radiation does deposited in the tumour is the same for all patients. Novel treatment opportunities, such as Intensity Modulated Radiation Treatment, will require more advanced molecular imaging probes to allow the RT approach to be personalized. One clinical problem is related to the tumour delineation and the differnetiation of dose within the tumour. Thirdly, there is also a need to reduce RT dose to the normal tissues in order to avoid negative side effects, specifically salivatory glands of the head. In some cases, the salivatory glands are non-functioning and if these cases could be detected as part of a routine scan, RT dose planning does not need to avoid the glands and a higher radiation does could be given to the toumour without increased side effects.

Discussion or citation of a reference herein shall not be construed as an admission that such reference is prior art to the present invention.

### SUMMARY OF THE INVENTION

In view of the long felt need for optimal staging of head and neck cancer, more advanced molecular imaging tracers to personalize RT and evaluation of salivary gland function for improving the curative outcome of RT in head and neck cancer, the present invention relates to both the development of a PET tracer that could be used as an imaging tracer for head and neck cancer and methods of imaging head and neck cancer. A pharmaceutical comprising the compound and a kit for the preparation of the pharmaceutical are also provided.

In one embodiment of the invention, a method for the in vivo diagnosis or imaging of a head and neck cancer in a subject is claimed that comprises administration of a PET tracer.

In another embodiment, a PET tracer for imaging head and neck cancer is disclosed wherein the PET tracer is ACE. The PET tracer can also be ¹⁸F-acetate.

In yet another embodiment comprises a pharmaceutical composition which comprises the compound of a PET tracer, together with a biocompatible carrier in a form suitable for mammalian administration.

In a further embodiment of the present invention comprises a kit of the compound of a PET tracer, or a salt or solvate thereof, wherein the kit is suitable for the preparation of the pharmaceutical composition.

Yet in another embodiment of the invention, a method for personalized RT treatment for head and neck cancer in a subject is claimed that comprises administering a pharmaceutical composition comprising a compound of a PET tracer, tracing tumor delineation and giving personalized radiation dose amount in the tumor.

The present invention also provides a method for personalized RT treatment for head and neck cancer in a subject is claimed that comprises administering a pharmaceutical composition comprising a compound of a PET tracer, evaluating salivary gland function and giving personalized radiation dose amount in the tumor.

### BRIEF DESCRIPTION OF THE FIGURES

FIG. 1 shows a head and neck cancer patient with squamous cell carcinoma in left tonsil with different imaging modalities/tracers. (a) CT, (b) FDG-PET, (c) fused FDG-PET, (d) ACE-PET, (e) fused ACE-PET.

FIG. 2 shows a head and neck cancer patient with squamous cell carcinoma in left base of the tongue and metastases at the right side of the neck with different imaging modalities/tracers. (a) CT, (b) FDG-PET, (c) fused FDG-PET, (d) ACE-PET, (e) fused ACE-PET.

FIG. 3 shows a lymph node metastasis at the right side of the neck in a head and neck cancer with different imaging modalities/tracers. (a) CT, (b) FDG-PET, (c) fused FDG-PET, (d) ACE-PET, (e) fused ACE-PET.

FIG. 4 shows the ratio of the ACE and FDG volumes of the primary tumors.

### DETAILED DESCRIPTION OF THE INVENTION

The present invention relates to examining patients with head and neck cancer by investigating optimal PET tracer uptake revealed through Positron Emission Tomography (PET) that has more optimal staging than computer tomography (CT), Magnetic Resonance Imaging tomography (MRI) and FDG-PET.

PET imaging is a tomographic nuclear imaging technique that uses radioactive tracer molecules that emit positrons. When a positron meets an electron, they both are annihilated and the result is a release of energy in the form of gamma rays, which are detected by the PET scanner. By employing natural substances that are used by the body as tracer molecules, PET does not only provide information about structures in the body but also information about the physiological function of the body or certain areas therein. Furthermore, the choice of a tracer molecule depends on what is being scanned. Generally, a tracer is chosen that will accumulate in the area of interest, or be selectively taken up by a certain type of tissue, e.g. cancer cells. Scanning consists of either a dynamic series or a static image obtained after an interval during which the radioactive tracer molecule enters the biochemical process of interest. The scanner detects the spatial and temporal distribution of the tracer molecule. PET also is a quantitative imaging method allowing the measurement of regional concentrations of the radioactive tracer molecule. Commonly used radionuclides in PET tracers are ¹¹C, ¹⁸F, ¹⁵O, ¹³N or ⁷⁶Br.

Furthermore, tracers labeled with short-lived positron emitting radionuclides (e.g. ¹¹C, t_{1/2}=20.3 min) are frequently used in various non-invasive in vivo studies in combination with PET. Because of the radioactivity, the short half-lives and the submicromolar amounts of the labeled substances, extraordinary synthetic procedures are required for the production of these tracers. An important part of the elaboration of these procedures is the development and handling of new ¹¹C- and ¹⁸F-labelled precursors. This is important not only for labeling new types of compounds, but also for increasing the possibility of labeling a given compound in different positions.

When compounds are labeled with ¹¹C, it is usually important to maximize specific radioactivity. In order to achieve this, the isotopic dilution and the synthesis time must be minimized. Isotopic dilution from atmospheric carbon dioxide may be substantial when [¹¹C] carbon dioxide is used in a labeling reaction. Due to the low reactivity and atmospheric concentration of carbon monoxide (0.1 ppm vs. 3.4×10⁴ ppm for CO₂), this problem is reduced with reactions using [¹¹C]carbon monoxide.

In the current invention, ACE and ¹⁸F-acetate are developed as optimal PET tracers for diagnosis of head and neck cancer. There are several advantages in using PET technique and optimal PET tracers in the diagnosis of head and neck cancer.

One advantage is that the methods of the instant invention provide optimal staging of this cancer is not reached in all patients using CT, MRI or FDG-PET. Another advantage is that the methods of the instant invention provide more advanced molecular imaging probes to allow the RT approach to be personalized thus opening doors for novel treatment opportunities, such as Intensity Modulated Radiation Treatment. Thirdly, in the cases where salivatory glands are non-functioning, the methods of the instant invention allows RT dose planning which does not need to avoid the glands and a higher radiation does could be given to the toumour without increased side effects.

After obtaining ACE and ¹⁸F-acetate, using an automated system termed FastLab or Tracerlab, high performance liquid chromatography (HPLC) is used to verify the structure of the analogues. A further tool was used to verify the structure of the analogues wherein a calculation study was conducted to look into the physical properties and 3D images of various analogues. The calculation study can be conducted using a computer-aided molecular design modeling tool also know as CAChe. CAChe enables one to draw and model molecules as well as perform calculations on a molecule to discover molecular properties and energy values. The calculations are performed by computational applications, which apply equations from classical mechanics and quantum mechanics to a molecule.

Below a detailed description is given of ACE and ¹⁸F-acetate that are suitable for use as an in vivo imaging agent for the diagnosis of head and neck cancer, as well as methods of imaging head and neck cancer. A pharmaceutical comprising the compound and a kit for the preparation of the pharmaceutical are also provided.

In one embodiment of the invention, a method for the in vivo diagnosis or imaging of a head and neck cancer in a subject is claimed that comprises administration of a PET tracer.

In another embodiment, a PET tracer for imaging head and neck cancer is disclosed wherein the PET tracer is ACE. The PET tracer can also be ¹⁸F-acetate.

Optimal staging of head and neck cancer is not reached in all patients using CT, MRI or FDG-PET. ACE and ¹⁸F-acetate detect more primary tumours and metastases than CT, MRI or FDG-PET and therefore provide a novel and improved solution to the current problem of non-optimal staging of head and neck cancer.

In yet another embodiment comprises a pharmaceutical composition which comprises the compound of a PET tracer, together with a biocompatible carrier in a form suitable for mammalian administration.

In a further embodiment of the present invention comprises a kit of the compound of a PET tracer, or a salt or solvate thereof, wherein the kit is suitable for the preparation of the pharmaceutical composition.

The kits comprise a suitable precursor of the second embodiment, preferably in sterile non-pyrogenic form, so that reaction with a sterile source of an imaging moiety gives the desired pharmaceutical with the minimum number of manipulations. Such considerations are particularly important for radiopharmaceuticals, in particular where the radioisotope has a relatively short half-life, and for ease of handling and hence reduced radiation dose for the radiopharmacist. Hence, the reaction medium for reconstitution of such kits is preferably a “biocompatible carrier” as defined above, and is most preferably aqueous.

A suitable kit container comprises a sealed container which permits maintenance of sterile integrity and/or radioactive safety, plus optionally an inert headspace gas (e.g. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired e.g. to change the headspace gas or degas solutions.

The kits may optionally further comprise additional components such as a radioprotectant, antimicrobial preservative, pH-adjusting agent or filler. By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation. The “biocompatible cation” and preferred embodiments thereof are as described above. By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution, i.e. in the radioactive imaging product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the conjugate is employed in acid salt form, the pH adjusting agent may optionally be provided in a separate vial or container, so that the user of the kit can adjust the pH as part of a multi-step procedure.

The term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The “biocompatible carrier” is a fluid, especially a liquid, in which the compound is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Furthermore, the pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. For radiopharmaceutical compositions, the pre-filled syringe may optionally be provided with a syringe shield to protect the operator from radioactive dose. Suitable such radiopharmaceutical syringe shields are known in the art and preferably comprise either lead or tungsten. The radiopharmaceuticals may be administered to patients for PET imaging in amounts sufficient to yield the desired signal, typical radionuclide dosages of 0.01 to 100 mCi, preferably 0.1 to 50 mCi will normally be sufficient per 70 kg bodyweight.

Yet in another embodiment of the invention, a method for personalized RT treatment for head and neck cancer in a subject is claimed that comprises administering a pharmaceutical composition comprising a compound of a PET tracer, tracing tumor delineation and giving personalized radiation dose amount in the tumor.

Using standard RT approaches, the radiation dose deposited in the tumor is the same for all patients. Novel treatment opportunities, such as Intensity Modulated Radiation Treatment, will require more advanced molecular imaging probes to allow the RT approach to be personalized. One clinical problem is related to the tumor delineation and the differentiation of dose within the tumour. The tumour volumes derived from ACE and ¹⁸F-acetate PET images are significantly larger than volumes from FDG-PET, which demonstrates that radiolabelled acetate provide better tumour delineation for RT than existing methods.

The present invention also provides a method for personalized RT treatment for head and neck cancer in a subject is claimed that comprises administering a pharmaceutical composition comprising a compound of a PET tracer, evaluating salivary gland function and giving personalized radiation dose amount in the tumor.

There is also a growing need to reduce RT dose to the normal tissues in order to avoid negative side effects, specifically salivatory glands of the head. In some cases, the salivatory glands are non-functioning and if these cases can be detected as part of routine scan, RT dose planning does not need to avoid the glands and a higher dose could be given to the tumour without increased side effects. ACE and ¹⁸F-acetate PET are valuable for the evaluation of salivary gland function. Incorporating this information into the dose planning algorithm increases the curative outcome of RT in head and neck cancer.

Another embodiment comprises a method for the in vivo diagnosis or imaging of a head and neck cancer in a subject, further comprising administration of a pharmaceutical composition comprising a PET tracer.

### EXAMPLES

The invention is further described in the following examples which are in no way intended to limit the scope of the invention.

Experimental Studies

The results of the study described below in ten patients indicate that ACE-PET is more sensitive for detection of primary tumors and metastases in head and neck squamous cell carcinoma, compared to FDG. Increased acetate uptake is a prominent feature of the primary tumors and lymph node metastases of head and neck squamous cell carcinomas in this study. ACE-PET provided diagnostic images of good quality and might be a more sensitive tool for staging of head and neck tumors than FDG-PET in a subset of cancer patients. The use of ACE-PET for tumor volume delineation resulted in 51% larger volumes than FDG-PET.

Patients

Ten consecutive patients (median age 56, range 18-77 years), where of eight males and two females with histologically confirmed squamous cell carcinoma of the head and neck, were included in the study. None of the patients suffered from diabetes. The patients had neither been treated with radiotherapy nor with chemotherapy prior to inclusion. The clinical characteristics including the stage and the location of the primary tumors are shown in Table 1. Conventional staging of the tumors was performed by CT (n=9), MRI (n=1), histopathology and clinical examination. Histological confirmation was obtained by guided biopsies in all the primary tumors and most metastatic sites. The metastases not verified with biopsies (n=5) were deemed malignant based on the combination of all the available information and included a three month follow up. All patients participating in the study provided informed consent. The study was accepted by the ethical committee of the participating hospital.

PET Imaging

In all patients, both FDG-PET and ACE-PET were performed before the radiotherapy treatment. ACE and FDG-PET scans were performed on the same or consecutive day, except for one patient where the two types of PET scanning were done five days apart. PET studies were carried out with a dedicated PET scanner (Siemens ECAT HR⁺, Knoxville, Tenn., USA) or with a PET/CT (GE Discovery S T, Milwaukee, Wis., USA). All patients were normoglycemic and were fasted at least 6 hours before tracer injection.

### Acetate PET Imaging

Six patients were studied with dedicated PET and four patients were investigated with PET/CT. A 32 minutes dynamic emission scan was performed immediately after intravenous injection of 10 MBq/kg body weight ACE. The scan time was 12×5s, 6×10s, 4×30s, 4×60s, 2×120s and 4×300s. Frame 30 (17-22 minutes after injection) generally provided the best image quality with highest tumor to background ratio and was therefore chosen for subsequent data analysis.

### FDG PET Imaging

Whole-body scanning was performed one hour after intravenous injection of 5 MBq/kg body weight FDG. Six patients were examined by PET/CT and four patients were studied by PET alone. The patients were instructed to remain recumbent and avoid voicing and other uses of neck muscles during the uptake period.

### Data Analysis

PET images were co-registered with the CT or MRI images in all patients by a normalized mutual information procedure supported by manual correction using Hermes Multimodality™ software (Nuclear Diagnostics, Stockholm, Sweden). FDG-PET and ACE-PET images were analyzed both qualitatively and quantitatively, using Hermes Volume Display™ version V2β. In qualitative analysis, PET images were interpreted visually by two nuclear medicine physicians and any disagreement was resolved by consensus. The tumor uptake of FDG and ACE was graded into negligible, mild, moderate and intensive compared to the contra-lateral or surrounding tissues. An abnormal uptake equal to or exceeding mild was considered positive. In quantitative analysis, the mean standardized uptake value (SUV) and tumor volumes delineated by ACE and FDG-PET were evaluated. SUV was calculated as mean radioactivity concentration in the volumes (Bq/cc) divided by injected dose (Bq) per kilogram body weight. For lesions with negligible uptake, similar tumor volumes were drawn manually by visual correlated fusion images.

Each tumor volume in FDG-PET and ACE-PET was delineated automatically by tracing an isoactivity pixel value set to 50% threshold of the maximum radioactivity corrected for background. The background was measured from a separately drawn region of interest (ROI) adjacent but at safe distance from the tumor. The isoactivity pixel value of each volume was calculated as:

Isoactivity pixel value=(MPVₜᵤₘₒᵣ+APV_{background})×50%

MPV is the maximum pixel value and APV is the average pixel value of the background ROI. This approach takes into account the variable background activity, effectively cancels the effect of varying background uptake on tumor volume measurements and was found to be highly reproducible. In those cases where the tumor location was near to the salivary glands with normally high physiological uptake of ACE, the tumor volumes were adjusted manually based on the combined information of CT and PET. Only one primary tumor volume and five metastases needed manual adjustments due to this reason.

### Statistical Analysis

The relationship between FDG SW and ACE SUV was determined by Pearson's correlation coefficient. ANOVA test was used to compare the tracer uptake with histological cell differentiation. The differences between the FDG and ACE SUVs and volumes were analyzed by nonparametric Wilcoxon signed rank test. Volumes of metastases were presented by median±interquartile, since it did not show a normal distribution. A p value <0.05 was considered statistically significant. Calculations were performed by SPSS version 11.5.

### Results

### Primary

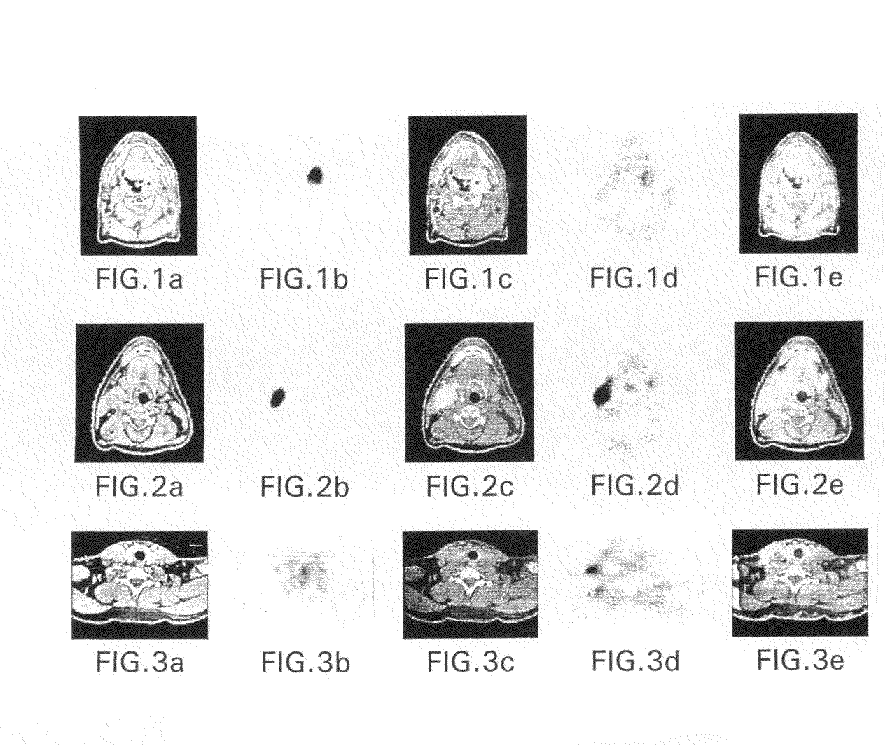

The qualitative and semi-quantitative comparison of the primary tumors in ACE-PET and FDG-PET are shown in Table 2. All of the primary tumors (10/10) were detected by ACE-PET, while nine of the ten lesions ( 9/10) were detected by FDG-PET and CT or MRI. PET and CT images are shown in FIG. 1 for one of the patients with cancer of the tonsil. The primary tumor of patient No 10 in the left base of the tongue could not be detected by either FDG-PET (SUV 1.9) or CT. ACE-PET, however clearly visualized the tumor with high uptake (SUV 3.7), see FIG. 2. One of the contra-lateral lymph node metastases was also visualized in this patient.

The range (mean±SD) of ACE SUV and FDG SUV was 2.5-10.6 (5.3±2.7) and 1.9-24.5 (9.6±7.0) respectively. FDG SUV tended to be higher than ACE SUV, although the difference was not statistically significant (p=0.07). No positive relation was found between ACE SLTV and FDG SUV (r=0.296, p=0.41). Furthermore, neither FDG SUV nor ACE SUV correlated with the histological grade of the cell differentiation (p=0.44 and p=0.81, respectively).

### Metastases

A total 21 metastatic lesions were detected in seven patients, see Table 3. Twelve of 21 lesions (12/21) were visualized by all used techniques. Almost all 20/21 lymph node metastases were detected by ACE-PET. The only false negative lesion in ACE-PET had a volume of 0.8 cc. This small lesion had an increased uptake in FDG-PET (SUV 3.8) and was also visualized with CT. Thirteen of 21 lesions were true positive by FDG-PET, whereas eight lymph node metastases in three patients were false negative; 16/21 metastases were true positive by CT or MRI, while five lesions in two patients were false negative (data not shown). In patient No 10, four lesions were false negative by both FDG-PET and CT, but all of them were true positive by ACE, see FIG. 3. The range of ACE SUV was 2.4-6.2 (4.0±1.3) and FDG SUV 0.9-10.0 (4.47±3.3) respectively. No significant difference (p=0.52) or correlation (r=0.383, p=0.09) were found between FDG SUV and ACE SUV.

High physiologic uptake was found in the salivary glands and tonsils, and the images displayed a lower ratio of uptake in tumor to background compared to FDG.

### Volumes

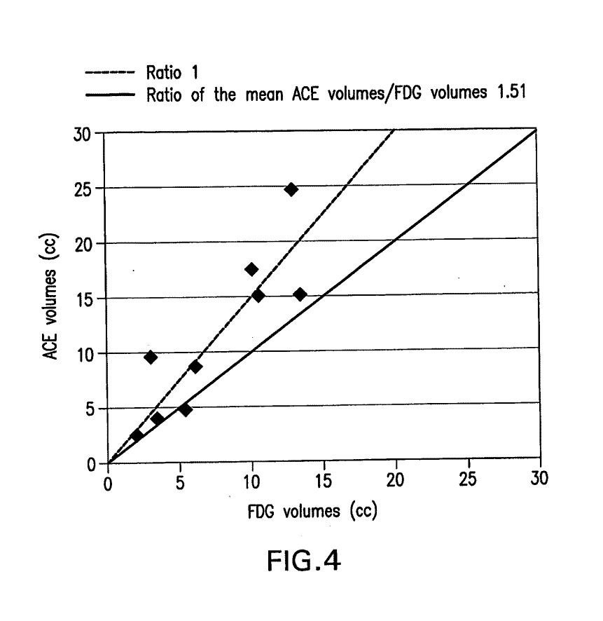

The calculated volumes of the primary tumors and metastases delineated by both ACE-PET and FDG-PET are shown in Table 2 and 3, respectively. The mean primary tumor volumes derived from ACE-PET were 11.2±7.4 cc (range 1.8-24.6 cc, n=9) compared to 7.4±4.5 cc (range 1.5-13.5 cc, n=9) for FDG-PET. The mean ACE-PET volumes were thus 51% larger than the volumes delineated by FDG-PET (p=0.02). Specially, in patient No 1, the ACE volume of the primary was three times larger than the corresponding FDG volume. The same relation for patient No 4 was almost a factor of two. Only in patient No 7 was the ACE delineated tumor volume smaller than the FDG volume. The ratio of ACE volumes to FDG volumes hence exceeded unity in nine of the ten patients, see FIG. 4.

Volumes of metastases were also larger (23%, p=0.005) drawn by ACE compared to FDG. The median volumes of lymph node metastases drawn by ACE were 2.9±10.3 cc, compared to the slightly lower values when FDG was used 2.3±7.4 cc.

TABLE 1
Patient clinical characteristics.

| Patient | Sex | Age(year) | Stage | Location | Histology diff |
|---|---|---|---|---|---|
| 1 | M | 77 | T4N2cM0 | Larynx | Low |
| 2 | F | 57 | T2N0M0 | Nose | Moderate |
| 3 | M | 59 | T2N0M0 | Nose | High |
| 4 | M | 53 | T3N0M0 | Nose/sinus | Low |
| 5 | F | 67 | T4N3M1 | Tonsilla | Low |
| 6 | M | 59 | T3N1M0 | Tonsilla | Low |
| 7 | M | 47 | T4N3M0 | Epipharynx | Low |
| 8 | M | 64 | T2N2aM0 | Tonsilla | Low |
| 9 | M | 18 | T3N3M0 | Epipharynx | Low |
| 10 | M | 45 | T2N2bM0 | Tongue base | High |

TABLE 2
Qualitative and semi-quantitative comparison between
ACE and FDG-PET for the primary tumors.

| | ACE-PET | | | FDG-PET | | |
|---|---|---|---|---|---|---|
| Patient | SUV | Visual | Volumes (cc) | SUV | Visual | Volumes (cc) |
| 1 | 9.2 | +++ | 9.5 | 13.5 | +++ | 3.1 |
| 2 | 2.5 | + | 3.9 | 3.8 | ++ | 3.4 |
| 3 | 6.0 | +++ | 8.6 | 24.5 | +++ | 6.0 |
| 4 | 2.7 | + | 24.6 | 4.4 | ++ | 13.0 |
| 5 | 5.7 | +++ | 17.5 | 12.9 | +++ | 10.1 |
| 6 | 3.8 | ++ | 15.0 | 7.9 | +++ | 10.5 |
| 7 | 10.6 | +++ | 4.7 | 6.7 | +++ | 5.3 |
| 8 | 4.9 | ++ | 1.8 | 4.7 | ++ | 1.5 |
| 9 | 3.9 | ++ | 15.1 | 15.4 | +++ | 13.5 |
| 10 | 3.7 | ++ | 1.8 | 1.9 | − | ¤ |
| Mean ± SD | 5.3 ± 2.7 | | 11.2 ± 7.4 | 9.6 ± 7.0 | | 7.4 ± 4.5 |

TABLE 3
Qualitative and semi-quantitative comparison
between ACE and FDG-PET for metastases.

| | Number | | | | | | |
|---|---|---|---|---|---|---|---|
| | of | ACE-PET | | | FDG-PET | | |
| Pa- | Meta- | | | Volumes | | | Volumes |
| tient | stasis | SUV | Visual | (cc) | SUV | Visual | (cc) |
| 1 | 1 | 5.9 | +++ | 1.2 | 1.5 | − | ¤ |
| | 2 | 5.9 | +++ | 2.2 | 5.2 | +++ | 1.4 |
| | 3 | 5.9 | +++ | 1.8 | 5.4 | +++ | 0.8 |
| | 4 | 4.7 | ++ | 1.9 | 4.9 | ++ | 1.7 |
| 5 | 1 | 5.1 | +++ | 101.0 | 13 | +++ | 82.4 |
| | 2 | 3.1 | ++ | 1.4 | 3.8 | ++ | 1.7 |
| 6 | 1 | 2.9 | ++ | 1.4 | 3.6 | ++ | 1.2 |
| 7 | 1 | 4.5 | ++ | 1.5 | 1.7 | − | ¤ |
| | 2 | 3.9 | ++ | 1.5 | 1.3 | − | ¤ |
| | 3 | 4.6 | ++ | 1.7 | 1.5 | − | ¤ |
| 8 | 1 | 5.8 | +++ | 4.6 | 7.6 | +++ | 3.9 |
| 9 | 1 | 3.0 | − | ¤ | 3.8 | ++ | 0.8 |
| | 2 | 2.8 | + | 1.8 | 6.4 | +++ | 1.5 |
| | 3 | 2.6 | + | 3.5 | 6.2 | +++ | 2.9 |
| | 4 | 3.7 | ++ | 14.5 | 8.1 | +++ | 13.2 |
| | 5 | 3.0 | ++ | 4.8 | 5.7 | +++ | 3.7 |
| 10 | 1 | 6.2 | +++ | 14.5 | 10.0 | +++ | 10.5 |
| | 2 | 2.4 | + | 1.1 | 1.1 | − | ¤ |
| | 3 | 2.6 | + | 1.6 | 1.2 | − | ¤ |
| | 4 | 2.7 | + | 0.8 | 0.9 | − | ¤ |
| | 5 | 3.5 | ++ | 1.8 | 1.0 | − | ¤ |

### Table and Figure Legends

Table 1. Patient clinical characteristics. M=male, F=female, diff=cell differentiation.

Table 2. Qualitative and semi-quantitative comparison between ACE and FDG-PET for primary tumors. SUV=standardized uptake value; −=negligible uptake, +=mild uptake, ++=moderate uptake, +++=intensive uptake; SD=standard deviation; ¤=not measurable.

Table 3. Qualitative and semi-quantitative comparison between ACE and FDG-PET for metastases. SUV=standardized uptake value; −=negligible uptake, +=mild uptake, ++=moderate uptake, +++=intensive uptake; ¤=not measurable. The median volumes of the metastases drawn by ACE were 2.9±10.3 cc, compared to 2.3±7.4 cc when FDG was used.

FIG. 1. Patient No 6 with squamous cell carcinoma in left tonsil. (a) CT, (b) FDG-PET, (c) fused FDG-PET, (d) ACE-PET, (e) fused ACE-PET. The tumor exhibited increased uptake of FDG (SUV 7.9) and ACE (SUV 3.8).

FIG. 2. Patient No 10 with squamous cell carcinoma in left base of the tongue and metastases at the right side of the neck. (a) CT, (b) FDG-PET, (c) fused FDG-PET, (d) ACE-PET, (e) fused ACE-PET. ACE-PET clearly exhibited high uptake in the primary tumor with SUV 3.7. However, FDG failed to show a significantly increased uptake with SUV only 1.9 and missed the primary tumor. CT has also showed a false negative result. All of the images showed the contra-lateral lymph node metastasis.

FIG. 3. A lymph node metastasis at the right side of the neck in patient No 10. (a) CT, (b) FDG-PET, (c) fused FDG-PET, (d) ACE-PET, (e) fused ACE-PET. The metastasis displayed increased uptake of ACE (SUV 3.5). But FDG-PET was false negative with no increased uptake (SUV 1.0). CT has also missed this lymph node metastasis.

FIG. 4. The ratio of the ACE and FDG volumes of the primary tumors. Nine of the ten volume ratios between ACE and FDG exceeded unity.

crc per instructions from thr PTO.

### Specific Embodiments, Citation of References

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. A method for the in vivo diagnosis or imaging of a head and neck cancer in a subject, comprising administration of a PET tracer.

2. A method of claim 1, wherein the PET tracer is ACE.

3. A method of claim 1, wherein the PET tracer is ¹⁸F-acetate.

4. A method for the in vivo diagnosis or imaging of a head and neck cancer in a subject, comprising administration of a pharmaceutical composition of a PET tracer.

5. A method of claim 4, wherein the pharmaceutical composition comprises the PET tracer, together with a biocompatible carrier in a form suitable for mammalian administration.

6. A kit comprising the PET tracer, or a salt or solvate thereof, wherein said kit is suitable for the preparation of a pharmaceutical composition of claim 4.

7. A method for personalized RT treatment for head and neck cancer in a subject comprising administering a pharmaceutical composition of a compound of a PET tracer, tracing tumor delineation and giving personalized radiation dose amount in the tumor.

8. A method for personalized RT treatment for head and neck cancer in a subject comprising administering a pharmaceutical composition of a compound of a PET tracer, evaluating salivary gland function, and giving personalized radiation dose amount in the tumor.
